(19) United States
(12) Patent Application Publication
(10) Pub. No.: US 2010/0072399 A1
(43) Pub. Date: Mar. 25, 2010

(54) Portable Photodynamic Disinfection Light Delivery Device for Catheter

(75) Inventors: Cale Street, Edmonds, WA (US); Pierre LeDuc, White Rock (CA); Carol Leacy, Vancouver (CA); Marcus Alan Latham, Seattle, WA (US)

Correspondence Address: DOBRUSIN & THENNISCH PC, 29 W LAWRENCE ST, SUITE 210, PONTIAC, MI, 48342, US

(73) Assignee: Ondine International Holdings Ltd.

(21) Appl. No.: 12/564,476

(22) Filed: Sep. 22, 2009

Related U.S. Application Data
(60) Provisional application No. 61/099,239, filed on Sep. 23, 2008.

(51) Int. Cl. A61L 2/10; A61L 2/08

(52) U.S. Cl. 250/492.1; 250/494.1; 250/504 R

(57) ABSTRACT

The present invention provides a portable photodynamic disinfection light delivery device comprising: two opposing light sources, an energy source to supply energy to the light sources, and a housing for receiving the two light sources and the energy source; wherein (i) the housing includes two members each having a clamp portion for receiving one of the two light sources and an attachment mean connecting the clamp portions; (ii) the clamp portions during photodynamic disinfection surround a catheter disinfection site; and (iii) the two light sources together provide circumferential illumination at least one wavelength that can activate at least one photosensitizer so as to reduce microbes located at the catheter disinfection site. The present invention provides a catheter disinfection method comprising: applying circumferential illumination at a wavelength that activates a photosensitizer located at a catheter disinfection site so as to reduce microbes located at the catheter disinfection site using the portable photodynamic disinfection light device described above.

### CLAIM OF BENEFIT OF FILING DATE

This application claims the benefit of the filing date of U.S. Provisional Application Ser. No. 61/099,239 titled: “PORTABLE PHOTODYNAMIC DISINFECTION LIGHT DELIVERY DEVICE FOR CATHETER DISINFECTION” filed on Sep. 23, 2008, and the filing date of PCT Application No. PCT/US09/57699 titled: “PORTABLE PHOTODYNAMIC DISINFECTION LIGHT DELIVERY DEVICE FOR CATHETER DISINFECTION” filed on Sep. 21, 2009, both applications incorporated herein by reference for all purposes.

### FIELD OF INVENTION

The present invention relates to a portable photodynamic disinfection light delivery device. More specifically, it relates to a portable photodynamic disinfection light delivery device that provides circumferential illumination at desired wavelength(s) for photodynamic disinfection of catheters and related components (e.g., catheter connectors, hubs, tubing, etc.).

### BACKGROUND OF INVENTION

Antibiotic resistant pathogens often colonize medical devices used in healthcare settings such as catheters, thereby creating a serious risk for infection. Several studies have shown that dangerous pathogens, including methicillin resistant Staphylococcus aureus (“MRSA”), can survive and be recovered from surfaces and devices in healthcare settings. See R. Huang, S. Mehta, D. Weed, C. S. Price (2006) Methicillin resistant Staphylococcus aureus survival on hospital fomites, Infection Control and Hospital Epidemiology 27(11):1267-1269; T. Shiomori, H. Miyamoto, K. Makishima, M. Yoshida, T. Fujiyoshi, T. Udaka, T. Inaba, N. Hiraki (2002) Evaluation of bedmaking-related airborne and surface methicillin resistant Staphylococcus aureus contamination, Journal of Hospital Infection 50(1):30-35; J. M. Boyce (2007) Environmental contamination makes an important contribution to hospital infection, Journal of Hospital Infection 65(Suppl. 2):50-54. These pathogens contribute significantly to the increased incidences of hospital acquired infections (“HAI”) including catheter-related bloodstream infections.

Central venous catheters (“CVC”) are the most commonly placed catheters. It is estimated that 15 million CVC are used annually in the United States in intensive care units alone. See L. A. Mermel (2000) Prevention of intravascular catheter-related infections, Annals of Internal Medicine 132: 391-402. It is also estimated that approximately 80,000 catheter-related bloodstream infections occur each year in intensive care units. The cost associated with each incidence of catheter-related bloodstream infection is estimated to be between $34,508 to $56,000. See J. B. Dimick, R. K. Pelz, R. Consunji, S. M. Swoboda, C. W. Hendrix, P. A. Lipsett (2001) Increased resource use associated with catheter-related bloodstream infection in the surgical intensive care unit, Archives of Surgery 136:229-234. Even more alarming, an estimated 250,000 cases of catheter-related bloodstream infections occur annually in the United States; with 12% to 25% mortality when all-hospital incidences (including the incidences in intensive care units) are considered. See D. M. Kluger, D. G. Maki (1999) The relative risk of intravascular device related bloodstream infections in adults, Abstracts of the 39th Interscience Conference on Antimicrobial Agents and Chemotherapy, San Francisco, Calif.: American Society for Microbiology, 514.

The two most common routes for catheter-related bloodstream infections are: (1) migration of pathogens from the skin surface through the catheter tract; and (2) pathogen colonization of the outer hub/connection of long-term indwelling catheters. Several approaches are currently being used to reduce catheter-related infections. For example, antimicrobial lock solutions (e.g., minocycline, EDTA, ethanol, taurolidine and citrate) have been studied extensively for use to prevent infections caused by indwelling catheters. See I. Raad, H. Hanna, T. Dvorak, G. Chaiban, R. Hacham (2007) Optimal antimicrobial catheter lock solution, using different combinations of minocycline, EDTA, and 25 percent ethanol, rapidly eradicates organisms embedded in biofilm, Antimicrobial Agents and Chemotherapy 51(1):78-83. A recent systematic review and meta-analysis looking at clinical use of antimicrobial lock solutions for prevention of catheter-related infections concluded that this approach reduces the incidences of catheter-related bloodstream infections. See D. Yahav, B. Rozen-Zvi, A. Gafter-Gvili, L. Leibovici, U. Gafter, M. Paul (2008) Antimicrobial lock solutions for the prevention of infections associated with intravascular catheters in patients undergoing haemodialysis: Systematic review and meta-analysis of randomized, controlled trials, Clinical Infectious Disease 47(1): 83-93.

The Center Disease Control has published the “Guidelines for the Preventions of Intravascular Catheter-Related Infections” recommending consideration of the material used to make catheters and connections, insertion site, and aseptic technique as variables influencing infection rate. See N. P. O'Grady, et. al (2002) Guidelines for the Prevention of Intravascular Catheter-Related Infections, MMWR Recommendations and Reports 51(RR10):1-26 (http://www.cdc.gov/mmwr/preview/mmwrhtml/rr5110a1.htm). This document also mentioned the use of catheters impregnated with antibiotics, chemical disinfection (e.g., chlorhexidine), and silver-based antimicrobial.

Based upon the foregoing, a quick, effective, and environmentally friendly method of disinfecting medical devices including catheters, catheter connections and medical tubings in healthcare settings would be extremely valuable in reducing incidences of HAI.

### SUMMARY OF THE INVENTION

Photodynamic disinfection is the use of a photosensitizer activated by light or electromagnetic radiation of specific wavelength(s) to inhibit or eliminate microbes including pathogens. Photodynamic disinfection has been demonstrated to be an effective non-antibiotic antimicrobial approach in vitro. One major advantage of photodynamic disinfection is that it is not subject to issues of resistance that plague the use of antibiotics. Another advantage of photodynamic disinfection is the photosensitizer(s) used for photodynamic disinfection are biocompatible and environmentally friendly with minimal, if any, side effects; especially when they are used in low concentrations. Photodynamic disinfection can also be used to disinfect inanimate surfaces and medical devices such as catheters, catheter connections, medical tubing, or the like in healthcare settings thereby reducing the incidences of HAI's.

The present invention provides a portable photodynamic disinfection light delivery device comprising: two opposing light sources, an energy source to supply energy to the light sources, and a housing for receiving, containing, and/or housing (collectively hereinafter referred to as “receiving” the two light sources and the energy source; wherein (i) the housing includes two members each having a clamp portion for receiving one of the two light sources and an attachment mean connecting the clamp portions; (ii) the clamp portions during photodynamic disinfection surround a catheter disinfection site; and (iii) the two light sources together provide circumferential illumination at least one wavelength that can activate at least one photosensitizer so as to reduce microbes located at the catheter disinfection site. The catheter disinfection site is a desired disinfection site of a catheter or a catheter related component such as a catheter connector, hub, medical tubing, or the like. The device of the present invention may optionally further include a controller for controlling the energy source and the light sources. The controller is optionally received by the housing.

The present invention provides a catheter disinfection method comprising: applying circumferential illumination at a wavelength that activates a photosensitizer located at a catheter disinfection site so as to reduce microbes located at the catheter disinfection site using the portable photodynamic disinfection light device described above.

### BRIEF DESCRIPTION OF THE DRAWING

The present invention will be more clearly understood when considering the accompanying drawing of which:

FIG. 1 shows a prospective side view of a portable photodynamic disinfection light delivery device in closed configuration in accordance with the present invention in closed configuration;

FIG. 2 shows a prospective side view of the device of FIG. 1 in open configuration;

FIG. 3 shows a prospective view of the device of FIG. 1 in closed configuration enclosing a portion of a catheter including the catheter disinfection site;

FIG. 4 shows a prospective view of another embodiment of a portable photodynamic disinfection light delivery device in accordance with the present invention in open configuration;

FIG. 5 shows a prospective view of the device of FIG. 4 in open configuration with a portion of a catheter located within the device of FIG. 4;

FIG. 6 shows a prospective view of the device of FIG. 4 in closed configuration enclosing a portion of a catheter including the catheter disinfection site;

FIG. 7 shows a prospective side view of a catheter's luer lock having surfaces coated with photosensitizer;

FIG. 8 shows a prospective side view of two portions of a medical tubing in an unconnected fashion; and

FIG. 9 shows the medical tubing shown in FIG. 8 in a connected fashion.

### DESCRIPTION OF THE PREFERRED EMBODIMENT

A. Photodynamic Disinfection Light Delivery Device

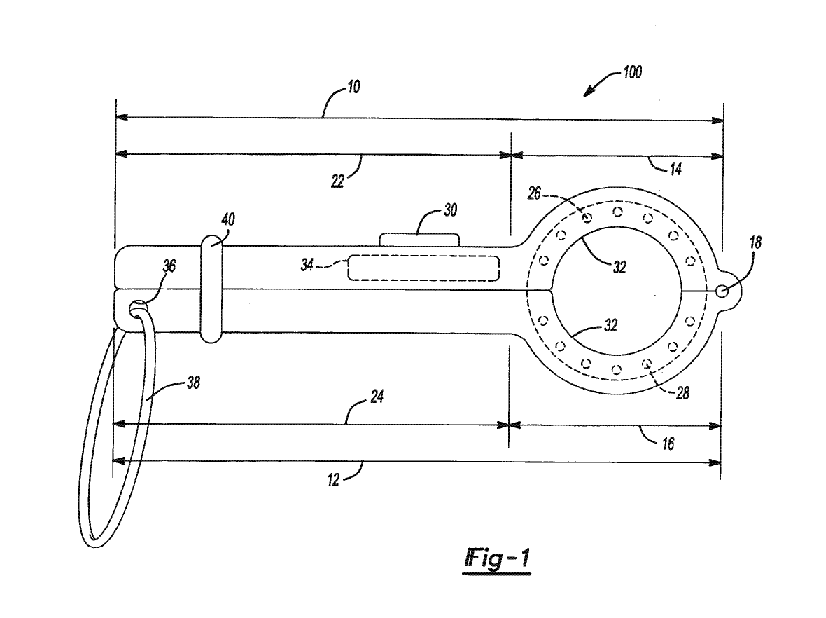

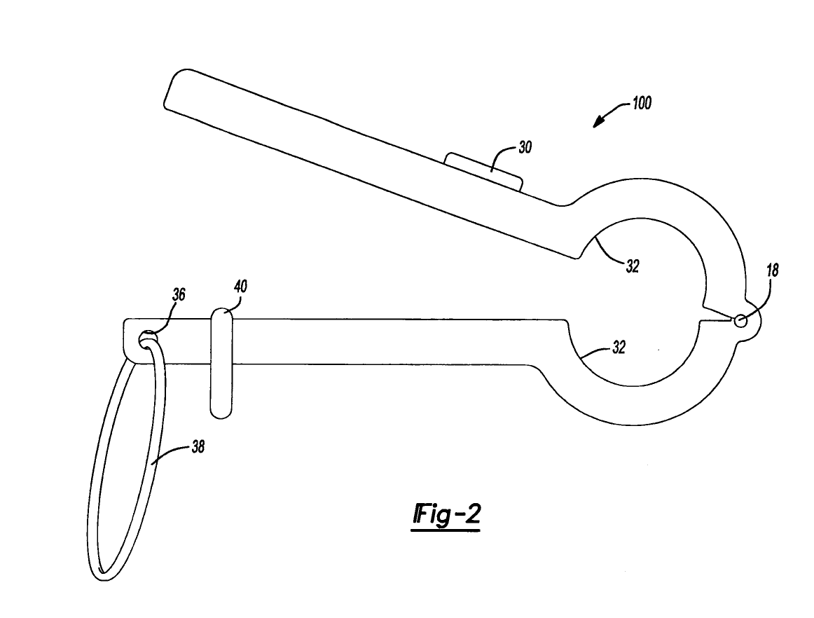

Referring to FIGS. 1-3, the present invention provides for a photodynamic disinfection light delivery device 100 comprising a housing 8 having two members 10, 12 each having a clamp portion 14, 16 that can be connected to each other with attachment means 18 to form a clamp that surrounds a catheter disinfection site 20. Each of the members 10, 12 may optionally have a handle portion 22, 24 that facilitates opening, closing, and handling of the device 100. Each clamp portion (14, 16) accepts a light source (26, 28) thereby allowing the light sources 26, 28 to be in opposing of each other (e.g., facing or opposing each other). The light sources 26, 28 provide desired wavelength(s) that activate at least one photosensitizer located at the catheter disinfection site 20 so as to reduce microbes located at the catheter disinfection site 20. “To reduce microbes” means for the purpose of this specification, to reduce, inhibit, eliminate, and/or decolonize microbes.

The catheter disinfection site 20 is a desired disinfection area of a catheter 21 as shown in FIG. 3 or a catheter related component such as a catheter connector, a catheter hub, a medical tubing, or the like. When the clamp portions 14, 16 form a clamp that surrounds the catheter disinfection site 20, the light sources 26, 28 apply circumferential illumination (i.e., light, electromagnetic radiation, or the like) at specific wavelength(s) to the catheter disinfection site 20. This circumferential illumination of specific wavelength(s) activate at least one photosensitizer located at the catheter disinfection site 20 so as to decolonize microbes located at the catheter disinfection site 20. The circumferential illumination may optionally also provide UV radiation to provide additional method of disinfection of the catheter disinfection site.

The microbes to be inhibited, eliminated and/or decolonized may include any and all disease-related microbes or pathogens such as virus, fungus, and bacteria including Gram-negative organisms, Gram-positive organisms or the like. Some examples of microbes include but are not limited to, Staphylococcus aureus, Methicillin-resistant Staphylococcus aureus (“MRSA”), Escherichia coli (“E. coli”), Enterococcus fecalis (“E. fecalis”), Pseudomonas aeruginosa, Aspergillus, Candida, etc.

The specific wavelength(s) include wavelengths selected from the continuous electromagnetic spectrum such as ultraviolet (“UV”), visible, and the infrared (near, mid and far). Exemplary ranges of wavelengths are between about 100 nm to 1,600 nm, between about 300 nm to about 900 nm, between about 450 nm to about 850 nm, between about 600 nm to about 700 nm, and between about 630 nm to about 670 nm. The wavelengths may vary depending upon the particular photosensitizing compound used and the light intensity. The light produced may be a single wavelength or multiple wavelengths.

The light sources 26, 28 are suitable art-disclosed light emitting devices such as lasers, light emitting diodes (“LEDs”), incandescent sources, fluorescent sources, or a combination thereof. For example, in one embodiment, each of the light sources 26, 28 is an array of laser diodes, LEDs, or a combination thereof. The output of the light sources 26, 28 is optionally adjustable and controlled by a suitable controller 30 (e.g., switch) so that the operator can modify the wavelength, the power output, the size of illumination, or combinations thereof while carrying out the present method. Alternatively, the controller 30 can simply be a switch that automatically turns (i) on the light sources 26, 28 when the clamp portions 14, 16 are clamped together and (ii) off when the clamp portions 14, 16 are not clamped together. In another alternative embodiment, the controller 30 can automatically turns (i) on the light sources 26, 28 when the presence of the catheter disinfection site 20 is detected by a sensor of the controller 30 and (ii) off when the presence of the catheter disinfection site 20 is not detected by a sensor of the controller 30. Furthermore, for example, the wavelength of a laser may be adjusted to activate different photosensitizers in the photosensitizing composition. Alternately, the power of the light sources 26, 28 may be increased or decreased after an application of light energy to the catheter disinfection site 20. In addition, the light sources 26, 28 may further include a temperature monitoring device so that overheating of the catheter disinfection site 20 may be avoided. Suitable temperature monitoring devices may comprise an IR device, a fiber optic device, a thermocouple, or a combination thereof.

The device 100 may optionally include a cover 32 over the light sources 26, 28 to prevent direct contact between the light sources 26, 28 and the catheter disinfection site 20. The cover 32 can be constructed out of any suitable optically transparent or translucent (hereinafter collectively referred to as “optically translucent”) material such as silicone, plastic, polymer, etc. For example, the cover 32 can be constructed our of a soft and flexible polymer material, such as medical grade silicone.

The device 100 further includes an energy source 34 to power the light sources 26, 28. The (optional) controller 30 controls the energy source 34 (e.g., turning it on, off, etc.) and/or the light sources 26, 28. Suitable examples of the energy source 34 are alternating current source (“AC”) and direct current source (“DC”). For example, in one embodiment and as shown in FIG. 1, the energy source 34 is comprised of battery or batteries (e.g., regular or rechargeable) housed within one of the members 10, 12. If the energy source 34 is comprised of rechargeable battery or batteries, the device 100 may optionally include a recharging port (not shown) which would allow the device to connect and/or dock directing into a recharging station.

To facilitate the handling of the device 100, the device 100 may optionally include art-disclosed suitable strap attachment means 36 such as a cavity within at least one of the members 10, 12 as shown in FIG. 1 to accept a strap 38. Furthermore, the device 100 may optionally include art-disclosed suitable locking means 40 such a clip or a loop as shown in FIG. 1. The locking means 40 provides removably secured attachment of the two members.

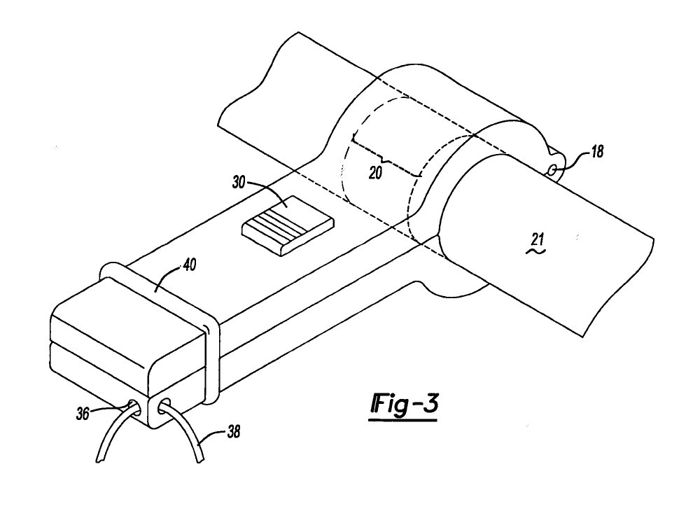

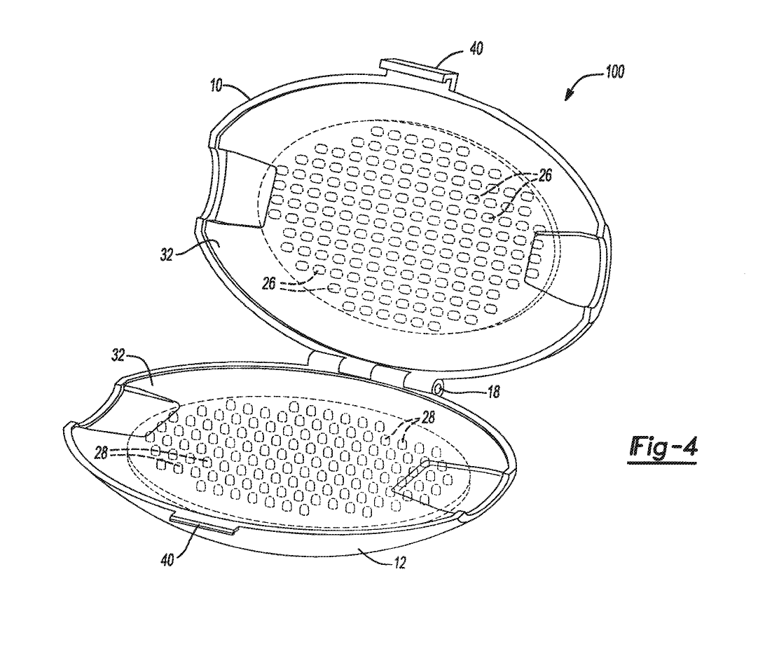

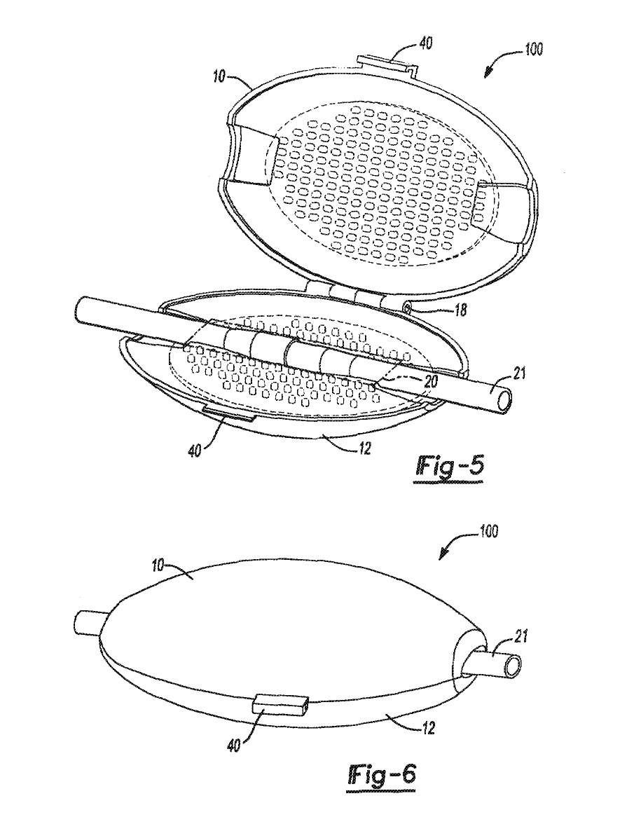

Referring to FIGS. 4-6, another embodiment of the device 100 is provided with the members 10, 12 connected together with the attachment means 18 in a clam like fashion with a clip as the locking means 40. The cover 32 is a sealed translucent polymer cushion and the light sources 26, 28 are arrays of LEDs. FIG. 4 shows the device 100 in an open configuration. FIG. 5 shows the device 100 in an open configuration with a portion of the catheter 21 located within the device 100 including the catheter disinfection site 20. FIG. 6 shows the device 100 in a closed configuration enclosing a portion of the catheter 21 including the catheter disinfection site 20.

B. Method for Photodisinfection of a Catheter or Related Component

The present invention includes a method for photodynamic disinfection of a catheter or related catheter component comprising applying a photosensitizer 42 to the catheter disinfection site 20 (as defined and described above). The photosensitizer 42 can be any suitable art-disclosed photosensitizer that has at least an antimicrobial action upon application of light or electromagnetic radiation of certain wavelength(s). Suitable classes of compounds that may be used as antimicrobial photosensitizers include tetrapyrroles or derivatives thereof such as porphyrins, chlorins, bacteriochlorins, phthalocyanines, naphthalocyanines, texaphyrins, verdins, purpurins or pheophorbides, phenothiazines, etc., such as those described in U.S. Pat. Nos. 6,211,335; 6,583,117; and 6,607,522 and U.S. Patent Publication No. 2003-0180224. Preferred phenothiazines include methylene blue (MB), toluidine blue (TBO), and those discussed in U.S. Patent Publication No. 2004-0147508. Other preferred antimicrobial photosensitizers include indocyanine green (ICG). Combinations of two or more photosensitizers, such as MB and TBO or the like, are also suitable. The photosensitizer 42 may be present in concentration of any suitable amounts. Examples are between about 0.001 percentage of total weight (wt %) and 10 wt %, between about 0.005 wt % and about 1 wt %, between about 0.01 wt % to about 0.5 wt %, and between about 0.02 wt % to about 0.1 wt %.

The photosensitizer 42 may also optionally include carriers, diluents, or other solvents for the photosensitizer 42 and may be used to adjust concentration of the photosensitizer 42. The photosensitizer 42 may be any suitable phase such as a liquid, gel, paste, putty, or solid (e.g., powder form). The photosensitizers mentioned above are examples and are not intended to limit the scope of the present invention in any way.

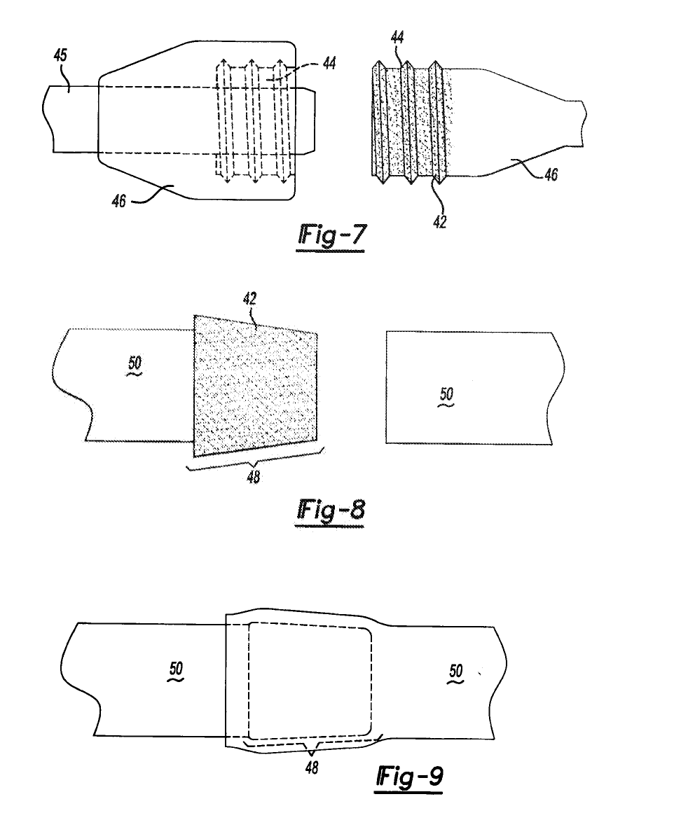

The photosensitizer 42 can be applied to the catheter disinfection site 20 in various fashions. For example, the photosensitizer 20 can be coated onto or impregnated into the catheter disinfection site 20 during or subsequent to the manufacturing process. Referring to FIG. 7 which shows a side view of a luer lock comprising two members 46 that are removably attached to each other with at least one of the two members connected to a medical tubing 45. The photosensitizer 2 is coated onto the connecting surfaces 44 of the two members 46. FIG. 7 shows only one of the connecting surfaces 44 is coated with the photosensitizer 42; but the present invention is not limited to coating just one of the connecting surfaces 44 and includes coating one or more of the connecting surfaces 44 of the luer lock. Referring to FIGS. 8-9, a tip 48 of a friction fit medical tubing 50 is coated with the photosensitizer 42. FIG. 8 shows the medical tubing 50 in a side view not connected and FIG. 9 shows the medical tubing 50 in a side view connected.

The method of the present invention further includes placing the device 100 described above around the catheter disinfection site 20 and applying light delivered by the device 100 to catheter disinfection site 20 at a wavelength absorbed by the photosensitizer so as to inhibit or eliminate microbes located in the catheter disinfection site. The method may optionally further include applying circumferential illumination using the device 100 at least one UV wavelength to provide additional UV disinfection of the catheter disinfection site.

Although there has been hereinabove described a portable photodisinfection light delivery device and method for photodisinfection of the catheter disinfection in accordance with the present invention, for purposes of illustrating the manner in which the invention may be used to advantage, it will be appreciated that the invention is not limited thereto. Accordingly, any and all modifications, variations, or equivalent arrangements which may occur to those skilled in the art should be considered to be within the scope of the present invention as defined in the appended claims.

What is claimed is:

1. A portable photodynamic disinfection light delivery device comprising:
Two opposing light sources;
an energy source to supply energy to the two light sources; and
a housing for receiving the two light sources and the energy source;
wherein
(i) the housing includes two members each having a clamp portion for receiving one of the two light sources and an attachment mean connecting the clamp portions;
(ii) the clamp portions during photodynamic disinfection surround a catheter disinfection site; and
(iii) the two light sources together provide circumferential illumination at least one wavelength that can activate at least one photosensitizer so as to inhibit microbes located at the catheter disinfection site.

2. The device according to claim 1 further comprising a controller for controlling the energy source and the light sources and the controller is received by the housing.

3. The device according to claim 2 wherein the controller is a switch that automatically turns the light sources and the energy source on when the clamp portions are clamped together and off when the light sources and the energy source are not clamped together.

4. The device according to claim 2 further comprising a sensor that detects presence of the catheter disinfection site placed within the device and communicates with the controller to automatically turn the light sources and the energy source on when the presence of the catheter disinfection site is detected by the sensor and off when the presence of the catheter disinfection site is not detected by the sensor.

5. The device according to claim 2 wherein the controller controls the at least one wavelength.

6. The device according to claim 1 further comprising a temperature monitoring device that controls the light sources in order to avoid overheating of the catheter disinfection site.

7. The device according to claim 1 further comprising a cover is placed above each of the two light sources wherein the cover provides separation between each of the two light sources and the catheter disinfection site.

8. The device according to claim 7 wherein the cover is constructed of a flexible, optically translucent material.

9. The device according to claim 7 wherein the cover is constructed of silicone.

10. The device according to claim 1 wherein the housing further includes a strap attachment means.

11. The device according to claim 10 wherein the strap attachment means is a through-hole within the housing.

12. The device according to claim 1 wherein each of the two members includes a handling portion.

13. The device according to claim 1 wherein the device further includes locking means that removably secure the two members together.

14. The device according to claim 1 wherein at least one of the two light sources is an array of LEDs.

15. The device according to claim 1 wherein at least one of the two light sources is an array of laser diodes.

16. The device according to claim 1 wherein the two light sources also further provide circumferential illumination in at least one UV wavelength to provide additional UV disinfection of the catheter disinfection site.

17. The device according to claim 1 wherein the energy source includes at least one rechargeable battery and the device further includes a recharging port that connects to a recharging station and allows the at least one rechargeable battery to be recharged.

18. A catheter disinfection method comprising: applying circumferential illumination at a wavelength that activates a photosensitizer located at a catheter disinfection site so as to reduce microbes located at the catheter disinfection site using a portable photodynamic disinfection light delivery device comprising: two light sources; an energy source to supply energy to the two light sources; a controller for controlling the energy source and the light source; and a housing for receiving the two light sources and the energy source; wherein (i) the housing includes two members each having a clamp portion for receiving one of the two light sources and an attachment mean connecting the clamp portions; (ii) the clamp portions during photodynamic disinfection surround a catheter disinfection site; and (iii) the two light sources together provide the circumferential illumination at a wavelength that activates a photosensitizer located at a catheter disinfection site so as to reduce microbes located at the catheter disinfection site.

19. The method according to claim 18 wherein the portable photodynamic disinfection light delivery device further includes a controller for controlling the energy source and the light sources and the controller is received by the housing.

20. The method according to claim 18 further comprising of applying circumferential illumination at least one UV wavelength using the device to provide additional UV disinfection of the catheter disinfection site.
